(19) United States
(12) Patent Application Publication
(10) Pub. No.: US 2014/0296592 A1
(43) Pub. Date: Oct. 2, 2014

(54) PROCESS FOR THE FLUID CATALYTIC CRACKING OF OXYGENATED HYDROCARBON COMPOUNDS FROM BIOLOGICAL ORIGIN

(71) Applicant: SHELL OIL COMPANY, Houston, TX (US)

(72) Inventors: Wei ZHU, DongYing (CN); Yinsuo CAI, Dong Ying (CN); YiBin LIU, DongYing (CN); YongShan TU, Qingdao (CN); ChaoHe YANG, Qingdao (CN); Yunying QI, Sugar Land, TX (US); Robert Alexander LUDOLPH, Sugar Land, TX (US); James Lloyd JENKINS, Houston, TX (US); Theodorus Johannes BROK, Amsterdam (NL); Colin John SCHAVERIEN, Amsterdam (NL); Binghui LI, Sugar Land, TX (US)

(73) Assignee: SHELL OIL COMPANY, Houston, TX (US)

(21) Appl. No.: 14/227,621

(22) Filed: Mar. 27, 2014

(30) Foreign Application Priority Data
Mar. 28, 2013 (CN) 201310105557.7

(51) Int. Cl. C07C 7/10; C07C 4/06

(52) U.S. Cl. CPC C07C 7/10; C07C 4/06; C07C 2529/89
USPC 585/310

(57) ABSTRACT

A process for the fluid catalytic cracking of oxygenated hydrocarbon compounds from biological origin. The process comprises (a) contacting a feed comprising the oxygenated hydrocarbon compounds with a fluid catalytic cracking catalyst at elevated temperature to produce a cracked products stream, the feed comprising an amount of sulphur; (b) separating catalyst from the cracked products stream; (c) separating a light fraction from the cracked products stream; and (d) removing hydrogen sulphide from the light fraction by means of an amine treating process. The fluid catalytic cracking process involves the presence or use of water and/or steam and comprises a working-up process of the cracked products stream. In the working-up process, one or more chemical additives for reducing or hindering the formation of foam in amine liquids selected from defoamers and demulsifiers are added to the amine solvent in one or more amine treaters.

### PRIORITY CLAIM

The present non-provisional application claims priority from Chinese application no. 201310105557.7, filed Mar. 28, 2013, the disclosures of which are incorporated herein by reference.

### TECHNICAL FIELD

The present disclosure relates to a process for the fluid catalytic cracking of oxygenated hydrocarbon compounds from biological origin.

### BACKGROUND

Fluid catalytic cracking (FCC) is an important conversion process in present oil refineries. It is especially used to convert high-boiling hydrocarbon fractions derived from crude oils into more valuable products such as gasoline components (naphtha), fuel oils and (olefinic) gases (ethene, propene, butene, LPG). The feedstocks for the FCC process are high boiling oil fractions.

With the diminishing supply of crude mineral oil, use of renewable energy sources is becoming increasingly important for the production of liquid fuels. These fuels from renewable energy sources are often referred to as biofuels. Accordingly, it would be desirable to have a process where part or all of the feed for an FCC unit comprising a material of biologic origin.

### SUMMARY

For that reasons, applicant started test-runs to establish whether or not part or all of the feed for a commercial FCC unit could be replaced by material of biologic origin, more especially oils and fats of plant or animal origin. During the test-runs it appeared that when changing the feed in a large (3000 barrels/day) integrated FCC unit from a completely crude mineral oil feed to a feed that comprises a certain amount of biofeed (in this case more especially 10 wt % of used cooking oil or 10 wt % of tallow oil) immediately problems occurred in the water/oil separation units and the amine treaters that are used to remove hydrogen sulphide from light product streams (dry gas and LPG). It appeared that emulsions were formed in the oil/water separators rather than the clear separation that was seen between the products from the catalytic cracking of crude oil feed only and the water fractions. In the amine treaters highly undesired stable foams, sometimes in combination with emulsions, were formed. These foams/emulsions are highly deleterious for the contact between the amine and the dry gas and/or LPG, which may result in insufficient removal of sour gasses. When the addition of biofeed was stopped, these problems disappeared.

It has now been found that the above described problems in an FCC process caused by the addition of biofeed to a crude oil based FCC feed may be overcome bythe addition of one or more chemical additives selected from demulsifiers and defoamers for separating oil/water emulsions into oil and water to one or more the oil/water separators and the addition of one or more chemical additives selected from defoamers and demulsifiers for reducing or hindering the formation of foam in amine liquids to the amine solvent in one or more of the amine treater. It appeared that the above problems could also be overcome by changing the operating conditions (emulsions could be removed from the separators, however, either these emulsions had to be burned or to be worked up in a separate process; the foaming could potentially be resolved by removing 5 to 10 vol %/day of the amine solution), however, the addition of demulsifiers and/or defoaming agents is much to be preferred over changed operation conditions (in view of costs and waste).

Thus, one aspect of the invention provides a process for the fluid catalytic cracking of oxygenated hydrocarbon compounds from biological origin. In one embodiment, the process comprises: (a) contacting a feed comprising the oxygenated hydrocarbon compounds with a fluid catalytic cracking catalyst at elevated temperature to produce a cracked products stream, the feed comprising an amount of sulphur; (b) separating catalyst from the cracked products stream; (c) separating a light fraction from the cracked products stream; and (d) removing hydrogen sulphide from the light fraction by means of an amine treating process. The fluid catalytic cracking process involves the presence or use of water and/or steam and comprises a working-up process of the cracked products stream. In the working-up process, one or more chemical additives for reducing or hindering the formation of foam in amine liquids selected from defoamers and demulsifiers are added to the amine solvent in one or more amine treaters.

In some embodiments, the oxygenated hydrocarbon compounds are derived from oil and fats from plant sources, animal sources or microbial sources, preferably tri-glycerides and/or free fatty acids. In some embodiments, the amount of oxygenated hydrocarbon compounds is up to 65 vol % of the total feed, preferably between 1 and 45 vol %, more preferably between 2 and 35 vol %, even more preferably between 3 and 25 vol %. In some embodiments, the amount of sulphur in the feed is up to 4 wt % based on total feed, preferably up to 3 wt %, more preferably between 0.1 and 2.5 wt %.

In some embodiments, in step (a) the elevated temperature is in the range of 300 to 750° C. and/or the contact time between the feed and the fluid catalytic catalyst is less than 10 seconds.

In some embodiments, the light fraction from the cracked products stream is a C1-C2 fraction or a C3-C4 fraction. In some embodiments, the light fraction from the cracked products stream is obtained by feeding separated cracked products stream to a distillation column, fractionating the cracked products stream into an offgas fraction comprising C1-C4 compounds and at least one further fraction, optionally followed by separating the fraction comprising the C1-C4 fraction into a fraction comprising C1-C2 compounds and a fraction comprising C3-C4 compounds. In some embodiments, the hydrogen sulphide is removed from a fraction comprising C1-C2 compounds and/or from a fraction comprising C3-C4 compounds.

In some embodiments, steam is added to the feed/fluid cracking catalyst and/or steam is used to improve the separation of the catalyst from the cracked products stream. In some embodiments, the light fraction is cooled down to obtain a cooled down gas stream and a liquid oil/water condensate, followed by separation of the oil and the water fraction in an oil/water separator. In some embodiments, the cooled down gas stream, before the further separation, is compressed to a pressure between 0.5 and 5 MegaPascal, whereafter the compressed gas stream is cooled down to obtain a cooled down gas stream and a liquid oil/water condensate, followed by separation of the oil and the water fraction in an oil/water separator. In some embodiments, an obtained fraction comprising C3-C4 compounds is cooled down to obtain a cooled down gas stream and a liquid oil/water condensate, followed by separation of the oil and the water fraction in an oil/water separator. In some embodiments, one or more chemical additives for separating oil/water emulsions into oil and water selected from demulsifiers and defoamers are added to the streams entering the oil/water separator or to the emulsions in the oil/water separator. In some embodiments, the chemical additive for separating oil/water emulsions into oil and water is chosen from (alkyl)phenol-formaldehyde resins, epoxy resins, amines, polyamines, amides, di-epoxides, alcohols, polyols, polyol block copolymers, and the alkoxylated, especially ethoxylated or propoxylated, derivatives there from.

In some embodiments, the one or more chemical additives for reducing or hindering the formation of foam in amine liquids selected from defoamers and demulsifiers are chosen from silicone compounds, EO/PO based polyglycols and high boiling alcohols. In some embodiments, the chemical additive is chosen from silicon based defoaming agents with the trade names NALCO EC9204, SAG 7133 and KS-604 or from polyglycol defoaming agents for use in aqueous systems with the trade names Maxamine 70B and Nalco EC 9079 A. In some embodiments, the one or more chemical additives selected from defoamers and demulsifiers for reducing or hindering the formation of foam in amine liquids are added to the streams entering the amine treater, to the amine solvent directly or to a make-up amine stream, or are sprayed onto the stable foam

In a preferred embodiment, there is provided a process for the fluid catalytic cracking of oxygenated hydrocarbon compounds from biological origin, the process comprising contacting a feed comprising the oxygenated hydrocarbon compounds with a fluid catalytic cracking catalyst at elevated temperature to produce a cracked products stream, the feed comprising an amount of sulphur, the process further comprising separating catalyst from cracked products stream, separating a light fraction from the cracked products stream and removing hydrogen sulphide from light fraction by means of an amine treating process, the fluid catalytic cracking process, including catalyst/product separation, involving the presence or use of water and/or steam, which process furthermore comprises the use of one or more oil/water separation steps in the working-up process of the cracked products stream, in which process one or more chemical additives for separating oil/water emulsions into oil and water selected from demulsifiers and defoamers are added to one or more oil/water separators or one or more chemical additives for reducing or hindering the formation of foam in amine liquids selected from defoamers and demulsifiers are added to the amine solvent in one or more amine treaters.

Other advantages and features of embodiments of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF PREFERRED EMBODIMENTS

The fluid catalytic cracking process (FCC) described herein is an important conversion process in present oil refineries. It is especially used to convert high-boiling hydrocarbon fractions of crude oils to more valuable products as gasoline components, fuel oils and (olefinic) gases (ethene, propene, butene, LPG). Modern FCC units are continuous processes that operate 24 hours a day for a period of two to four years. An extensive description of FCC technology is found in “Fluid Catalytic Cracking technology and operations”, by Joseph W. Wilson, published by PennWell Publishing Company (1997), “Fluid Catalytic Cracking; Design, Operation, and Troubleshooting of FCC Facilities” by Reza Sadeghbeigi, published by Gulf Publishing Company, Houston Tex. (1995) and “Fluid Catalytic Cracking technology and operations”, by Joseph W. Wilson, published by PennWell Publishing Company (1997).

The feedstocks for the FCC process, which feedstocks may also be used together with the biofeedstocks in embodiments of the present invention, are usually high boiling oil fractions, having a boiling point of at least 240° C., or even 320° C., suitably at least 360° C. or even at least 380° C. (at atmospheric pressure), e.g. a VGO or long residue. Usually heavy gas oils are used, or (high) vacuum gas oils. In addition, high boiling fractions from other refinery units, e.g. the thermal cracker, the hydrocracker and catalytic dewaxing units, may be used. The FCC feedstock above described may be obtained from a conventional crude oil (also sometimes referred to as a petroleum oil or mineral oil), an unconventional crude oil (that is, oil produced or extracted using techniques other than the traditional oil well method) or a renewable oil (that is, oil derived from a renewable source, such as pyrolysis oil or vegetable oil), a Fisher Tropsch oil (sometimes also referred to as a synthetic oil) and/or a mixture of any of these.

The oxygenated hydrocarbon compounds that can be used in embodiments of the present invention are biofeeds or biorenewable feedstocks. That is the feed is at least partially derived from a biological source such as, but not limited to, oil and fats from plant sources, including algae and seaweed, animal sources or microbial sources. Such feeds comprise primarily tri-glycerides and/or free fatty acids (FFA). Plant and animal oils and fats typically contain 0-30 wt % free fatty acids, which are formed during hydrolysis (e.g. enzymatic hydrolysis) of triglycerides. The amount of free fatty acids present in vegetable oils is typically 1-5 wt % and in animal fat, 10-25 wt %. Examples of these feedstocks include but are not limited to canola oil, corn oil, soy oil, castor oil, cottonseed oil, palm oil, sunflower oil, seaweed oil, tallow oil, fish oil, yellow and brown greases, and other oils of animal, vegetable or microbial origin. The tri-glycerides and FFA's contain aliphatic hydrocarbon chains in their structure having 9 to 22 carbons. Another example of a bio-renewable feedstock that can be used in the present invention is tall oil. Tall oil is a by-product of the wood processing industry. Tall oil contains esters and rosin acids in addition to FFA's. Rosin acids are cyclic carboxylic acids. For the process, the feed can include a single oil or a mixture of two or more oils, in any proportions. Triglycerides may be transesterified before use into alkylcarboxylic esters as formiates, acetates etc. Another biofeed may be pyrolysis oil (obtained by pyrolysis (destructive distillation) of biomass in a reactor at temperatures between 400 and 600° C.) or other liquid biocrudes.

Preferred biofeeds are liquid biofeeds, especially used cooking oil and tallow oil.

In embodiments of the present invention, in principle. the whole feed may be a biofeed. Suitably the amount of oxygenated hydrocarbon compounds is up till 65 vol % of the total feed, preferably between 1 and 45 vol %, more preferably between 2 and 35 vol %, even more preferably between 3 and 25 vol % or even between 4 and 15 vol %. The remaining part of the feed are conventional FCC feeds as described above.

The feed of embodiments of the present invention will contain a certain amount of sulphur. The sulphur may be present in the mineral part of the feed and/or in the biofeed, mainly, e.g. more than 70 wt % on total sulphur, or even more than 90 wt %, in the mineral part. The sulphur is present in the form of organic sulphur, e.g. sulphide, disulphides and aromatic sulphur compounds. The amount may be up to 6 wt % base on total feed, suitably the amount of sulphur in the feed is up till 4 wt %, preferably up till 3 wt %, more preferably between 0.1 and 2.5 wt %. Due to the reaction conditions during fluid catalytic cracking, the sulphur present in the feed is for a large part converted into hydrogen sulphide. Further, mercaptans will be produced.

The hydrogen sulphide ends up in the light product streams of the FCC process (especially dry gas and LPG). For a number of reasons (e.g. environmental reasons, oxidation problems, odor problems) the hydrogen sulphide needs to be removed from these products. It is possible to treat only one light product stream, e.g. the dry gas or the LPG stream, preferably both streams are treated. It is possible to treat only a fraction of a light product stream (e.g. dry gas or LPG), e.g. only 50 vol % or only 80 vol % of the product stream, but preferably the total light products stream is treated in the amine treater. An absorber in an amine treater usually has its own regenerator, it is also possible to use a common regenerator for a number of adsorbers.

The reactor, the regenerator and the main fractionator to be used in embodiments of the present invention are considered essential parts of the FCC unit. Preheated high boiling hydrocarbon feedstock comprising long-chain hydrocarbons, preheated usually to a temperature between 160 and 420° C., especially between 180 and 380° C., is injected into the reactor (riser reactor) where it is vaporized and cracked into smaller molecules by contacting and mixing with the very hot powdered catalyst from the regenerator. Often a recycle stream from the main fractionator is simultaneously injected into the reactor. Also (transport) steam may be injected into the riser reactor. The cracking reactions take place in the catalyst rising reactor within a period of between 0.3 and 12 seconds, especially between 0.6 and 5 seconds. The catalyst riser reactor usually is an elongated tubular reactor having a diameter between 0.2 and 2.5 m, often 0.5 to 1.5 meter. The length is usually between 8 and 32 m, often between 12 and 24 m. The reaction temperature in the riser reactor is usually between 460 and 610° C., the pressure between 0.1 and 0.3 MegaPascal (MPa). The catalyst/feed ratio is usually between 4 and 50, preferably between 5 and 35, more preferably between 6 and 20. The hydrocarbon vapors and/or transportation steam fluidize the powdered catalyst and the mixture of hydrocarbons and catalyst flows upwards through the riser reactor to enter a separation unit where the cracked hydrocarbons are separated from the “spent” catalyst particles. The separation process is usually carried out by a number of horizontal and/or vertical cyclones, often in two or more stages. Suitably at least 80 wt % of the full catalyst/product stream from the FCC process is further processed, preferably all catalyst/product stream is further processed. Usually at least 96% of the spent catalyst is removed from the cracked hydrocarbon stream, preferably 98%, more preferably 99%. The spent catalyst particles often flow down via a stripping unit in which by means of steam stripping product hydrocarbons are removed from the spent catalyst particles. From there the spent catalyst particles are sent to the regenerator unit. Since the cracking reactions produce an amount of carbonaceous material (often referred to as coke) that deposits on the catalyst, resulting in a quick reduction of the catalyst activity, the catalyst is regenerated by burning off the deposited coke with air blown into the regenerator. The amount of coke is usually between 2 and 10 wt % based on the feed. Hot flue gas leaves the top of the regenerator through one or more stages of cyclones to remove entrained catalyst from the hot flue gas. The temperature in the regenerator is usually between 640 and 780° C., the pressure between 0.15 and 0.35 MegaPascal (MPa). The residence time of the catalyst in the regenerator is usually between five minutes and 2 hours.

In a preferred embodiment of the present invention the elevated temperature to produce the cracked products stream is in the range of 300 to 750° C., especially 400 to 700° C., and the contact time between the feed and the fluid catalytic catalyst of less than 10 seconds, especially 0.5 to 8 seconds.

The catalytic cracking catalyst can be any catalyst known to the skilled person to be suitable for use in a cracking process. Preferably, the catalytic cracking catalyst comprises a zeolitic component. In addition, the catalytic cracking catalyst can contain an amorphous binder compound and/or a filler. Examples of the amorphous binder component include silica, alumina, titania, zirconia and magnesium oxide, or combinations of two or more of them. Examples of fillers include clays (such as kaolin).

The zeolite is preferably a large pore zeolite. The large pore zeolite includes a zeolite comprising a porous, crystalline aluminosilicate structure having a porous internal cell structure on which the major axis of the pores is in the range of 0.62 nanometer to 0.8 nanometer. The axes of zeolites are depicted in the ‘Atlas of Zeolite Structure Types’, of W. M. Meier, D. H. Olson, and Ch. Baerlocher, Fourth Revised Edition 1996, Elsevier, ISBN 0-444-10015-6. Examples of such large pore zeolites include FAU or faujasite, preferably synthetic faujasite, for example, zeolite Y or X, ultra-stable zeolite Y (USY), Rare Earth zeolite Y (=REY) and Rare Earth USY (REUSY). According to the present invention USY is preferably used as the large pore zeolite.

The catalytic cracking catalyst can also comprise a medium pore zeolite. The medium pore zeolite that can be used according to the present invention is a zeolite comprising a porous, crystalline aluminosilicate structure having a porous internal cell structure on which the major axis of the pores is in the range of 0.45 nanometer to 0.62 nanometer. Examples of such medium pore zeolites are of the MFI structural type, for example, ZSM-5; the MTW type, for example, ZSM-12; the TON structural type, for example, theta one; and the FER structural type, for example, ferrierite. According to the present invention, ZSM-5 is preferably used as the medium pore zeolite.

In embodiments of the present invention steam may be introduced in the process at a number of positions. Thus, steam may be introduced for instance at the lower end of the riser reactor, half way the riser reactor, in the stripper unit and in the transport pipe of spent catalyst to the regenerator. Steam is often added to the feed/fluid cracking catalyst and/or to the stripper unit to improve the separation of the catalyst from the cracked products stream. It is observed that often the feed to the FCC process may contain a certain amount of water.

The reaction product vapors obtained after the separation of the catalyst, generally at 400-660° C., especially 460-610° C., and 0.1 to 0.3 MegaPascal (MPa), and the vapors from the stripping unit flow to the lower section of the main fractionator, the product distillation column. Suitably at least 60 wt % of the products from the fluid catalytic process are introduced into the main fractionator, more suitably at least 80 wt %, preferably all products are introduced in the main fractionator. In the main fractionator the products are separated into the FCC end-products. Usually the main products are offgas (mainly C1-C4 hydrocarbons), naphtha, gasoline, light cycle oil, a heavier fraction suitable as fuel oil (sometimes two fractions are separated, light fuel oil and heavy fuel oil) and a heavy fraction. Some FCC units produce a light and a heavy naphtha fraction. The heaviest fraction, often referred to as slurry oil as it contains a certain amount of catalyst, is usually returned to the riser reactor. Also a part or all of one or more of the heavier fractions may be returned to the riser reactor.

The main fractionator offgas is generally cooled down, in which step a two phase liquid is formed, an oil phase containing the heavier hydrocarbon compounds and a water phase containing condensed water. Due to the presence of hydrogen sulphide, the water layer is often indicated as sour water. The gas/liquid stream is sent to a combined gas/oil/water separator, although also a separated gas/liquid and liquid/liquid separator can be used.

Preferably the light fraction, especially the fraction comprising C1-C4 compounds, is cooled down to obtain a cooled down gas stream and a liquid oil/water condensate, followed by separation of the oil and the water fraction in an oil/water separation step. (By a Cx compound is herein understood a compound containing x carbon atoms). The cooled down gas stream is sent to a gas recovery unit or gas concentration unit, usually to be separated into dry gas (mainly hydrogen, methane, ethane, ethene, nitrogen) and an LPG fraction (propane, propene, butane, butane). Optionally saturated and unsaturated compounds may be separated. Part or all of the off-gas stream (suitably 60 vol %, especially 80 vol %) may be sent to the gas recovery unit, preferably all off-gas is sent to the gas recovery unit. The gas fractions, and usually also the naphtha fractions, contain a certain amount of sulphur, mainly in the form hydrogen sulphide (gas fractions) or mercaptans (naphtha). To improve product specification and especially to prevent corrosion problems, the hydrogen sulphide (and, if present, also carbon dioxide) is removed, preferably through an amine absorption process. The amine treater usually will also remove at least a part of any mercaptans or sulphides present in the gas streams.

In the gas recovery unit the offgas is usually compressed (by the wet gas compressor) to a pressure between 0.5 and 5 Megapascal (MPa), preferably 1.0 to 2.5 Megapascal (MPa). This results, usually after cooling, in the formation of compressed gas and liquids. The gas and the liquids, an oily fraction comprising the heavier hydrocarbons and an aqueous fraction (sour water fraction), are usually separated in a combined gas/oil/water separator, although also a separated gas/liquid and liquid/liquid separator can be used. The compressed gas is sent to the lower section of an adsorber, often called the primary adsorber. Suitably a naphtha fraction of the main fractionator (usually unstabilized naphtha, i.e. containing low boiling compounds), is introduced in the upper section of the primary adsorber. Dry gas is obtained at the upper part of the adsorber. The dry gas is optionally introduced in the lower section of a so-called sponge adsorber, in which a lean oil is introduced at the top of the adsorber and rich oil (containing C3, C4+ compounds) is obtained at the lower part of the adsorber. In this way it is assured that the dry gas only contains C2 and lower molecules. The rich sponge oil may be regenerated and the regenerated light product stream may be introduced as feed in the primary adsorber. The liquid product of the primary adsorber is either directly or indirectly (via the gas/oil/water separator system) introduced in the upper part of a stripper column. In the stripper column any C1 or C2 compounds, and optionally some C3 compounds, are removed from the liquid fraction. The liquid fraction from the stripper column is usually sent to a debutanizer column, in which a C3-C4 fraction is separated from the FCC naphtha product (the stabilized FCC naphtha). Usually a liquid C3-C4 stream is obtained from the debutanizer column and a light, gaseous top fraction. After cooling, the light fraction will yield a gas fraction comprising light compounds and a two phase oil/water fraction. The cooled gas/liquid stream is sent to a combined gas/oil/water separator, although also a separated gas/liquid and liquid/liquid separator can be used. It is also possible to obtain a gaseous C4-minus top fraction from the debutanizer, which fraction is cooled down followed by separation of the three phases as described above.

It is observed that smaller and larger modifications of the above described product work-up are known and have been described in the literature.

In a preferred embodiment of the present invention the light fraction from the cracked products stream is a C1-C2 fraction or a C3-C4 fraction. In a preferred embodiment a C1-C2 and a C3-C4 fraction is obtained. Preferably hydrogen sulphide is removed from fraction comprising C1-C2 compounds and from fraction comprising C3-C4 compounds. Preferably the full C1-C2 fraction and the full C3-C4 fraction are subjected to the hydrogen sulphide removal process. Preferably hydrogen sulphide is removed from the full light product fraction as defined in the main claim.

Preferably 90 mol % of the hydrogen sulphide is removed from a product stream, preferably 96 mol %, more preferably 98 mol %, in a hydrogen sulphide removal process. A light fraction comprising C1-C4 compounds preferably comprises at least 75 mol % C1-C4 compounds based on hydrocarbon compounds, preferably 90 mol %. A light fraction comprising C1-C2 compounds preferably comprises at least 75 mol % C1-C2 compounds based on hydrocarbon compounds, preferably 90 mol %. A light fraction comprising C3-C4 compounds preferably comprises at least 60 mol % C3-C4 compounds based on hydrocarbon compounds, preferably 80 mol %.

Without wishing to be bound by any kind of theory, it is believed that the formation of stable foams in the amine gas treating process may be due to the presence of products from catalytically cracking triglycerides and/or catalytically cracking of free fatty acids. It is believed that even ppmv (parts per million by volume) of free fatty acids themselves may contribute to the foaming. The products from catalytically cracking triglycerides and/or catalytically cracking of free fatty acids and/or triglycerides may include free fatty acids which may be present in the light fraction. Without wishing to be bound by any kind of theory, it is therefore believed that the light fraction may further contain one or more products from catalytically cracking triglycerides and/or catalytically cracking of free fatty acids. For example the light fraction may further contain one or more oxygen containing C1-C4 compounds having a biological origin. Such oxygen containing C1-C4 compounds having a biological origin may suitably have a boiling point equal to or less than 64° C. at a pressure of 0.1 MegaPascal. Examples of such oxygen containing C1-C4 compounds having a biological origin include methanol, ethanol, propanol, butanol, formic acid, acetic acid, propionic acid, butanoic acid, acetone, formaldehyde, acetaldehyde and acrylaldehyde. In a preferred embodiment the light fraction is a fraction comprising one or more compounds from biological origin chosen from the group consisting of methanol, acetone, ethanol, propanol, butanol, formic acid, acetic acid, propionic acid, butanoic acid, formaldehyde, acetaldehyde and acrylaldehyde. More preferably the light fraction is a fraction comprising one or more compounds from biological origin chosen from the group consisting of acetone, acetic acid or propionic acid. Again, without wishing to be bound by any kind of theory, it is believed that due to the bio-feed in the FCC step, the concentration of such oxygen containing C1-C4 compounds in a light fraction may have increased compared to a conventional FCC feed and such increased concentration may lead to a different kind of foaming in an amine treating process.

In another preferred embodiment, the light fraction from the cracked products stream is obtained by feeding separated cracked products stream to a distillation column, fractionating the cracked products stream into an offgas fraction comprising C1-C4 compounds and at least one further fraction, optionally followed by separating fraction comprising the C1-C4 fraction into a fraction comprising mainly C1-C2 compounds (i.e. more than 80 mol % based on hydrocarbons) and a fraction comprising mainly C3-C4 compounds (i.e. more than 80 mol % based on hydrocarbons). Preferably at least two further fractions are obtained, more preferably at least four fractions.

In some embodiments of the invention, one or more demulsifying agents or defoaming agents are added to the streams entering the oil/water separator and/or to the emulsions in the oil/water separator. In principle, every compound that breaks emulsions can be used. Commercially available demulsifying/defoaming agents may be used. Such demulsifying agents are often intended to break emulsions of crude oil fractions and water, but may also be used in the specific application of the present invention. Preferably the demulsifying/defoaming agent is chosen from (alkyl)phenol-formaldehyde resins, epoxy resins, amines, polyamines, amides, di-epoxides, alcohols, polyols, polyol block copolymers, and the alkoxylated, especially ethoxylated or propoxylated, derivatives there from. Commercially available demulsifiers are typically a mixture of two to four different chemistries in a carrier solvent (e.g. xylene, (heavy) naphtha, isopropanol methanol, diesel etc.) For instance, products from the DEMTROL product range from DOW, the Tretolite product range of Baker Hughes or products from the Witbreak range from AKZO may be used.

In a preferred embodiment, one or more chemical additives for separating oil/water emulsions into oil and water selected from demulsifiers and defoamers are added to the streams entering the oil/water separator or to the emulsions in the oil/water separator. The amount of chemical additive is suitably up till 1 vol % of the liquid product stream, preferably up till 0.1 vol %, more preferably up till 0.01 vol %, the minimum amount being at least 1 vppm, preferably 20 vppm.

As indicated above, the offgas fraction contains a certain amount of sulphur, mainly in the form of hydrogen sulphide. As hydrogen sulphide is an undesired constituent of the gas fractions it is to be removed. This is suitably done by means of an amine treatment unit in which the gas stream is washed with an amine liquid that absorbs the hydrogen sulphide. The rich amine liquid is regenerated.

Amine gas treating, also known as gas sweetening or acid gas removal, refers to a process in which an aqueous solution of one or more alkylamines is used to remove hydrogen sulphide from a gas stream. In addition also carbon dioxide is removed. Amine gas treaters are especially used in refineries and natural gas processing plants. The most commonly used amines are monoethanolamine (MEA), diethanolamine (DEA), methyldiethanolamine (MDEA), diisopropanolamine (DIPA) and diglycolamine (DGA). Optionally also a physical solvent, e.g. sulfolan, may be present. The absorber and the regenerator are considered to be the main equipment pieces in the amine treater. In the absorber the downflowing amine solution absorbs hydrogen sulphide and carbon dioxide from the upflowing sour gas stream to produce a sweetened gas stream (no hydrogen sulphide/carbon dioxide) and an amine solution rich in the absorbed sour gasses. The resulting rich amine is then introduced in the top of the regenerator (a stripper with a reboiler) to produce regenerated or lean amine solution that is recycled to the absorber. The stripped overhead gas from the regenerator is concentrated hydrogen sulphide and carbon dioxide. Hydrogen sulphide rich gas is usually sent to a Claus process to recover the sulphur as elemental sulphur. The amine treating process has been described in Oilfield Processing of Petroleum, F. Manning and R. E. Thompson, PennWell Publishing Company, Tulsa, Okla.; Acid and Sour Gas Treating Processes, S. A. Newman (ed.), Gulf, 1985; Gas Purification, A. L. Kohl, R. B. Nielsen, Gulf Professional Publishing, 1997; EP 13049 and WO 2008/145680.

In the amine treater the absorber is usually operated at a relatively low temperature (suitably between 30 and 60° C.) and a relatively high pressure (suitably 0.5 to 15 Megapascal (MPa)) in order to absorb as much as possible of the acid gases in the amine liquid. The regenerator is usually operated at a relatively high temperature (suitably 110 to 130° C.) and a relatively low pressure (suitably 0.1 to 0.2 Megapascal (MPa) at the tower bottom) in order remove as much as possible of the acid gases from the amine liquid. In some cases a flash vessel may be used. Rich amine solution is introduced into the flash vessel at a pressure between the pressure of the absorber and the regenerator. Part of the absorbed gasses will come free here. The flashed amine solution is sent to the regenerator.

Preferably hydrogen sulphide is also removed from fraction comprising C3-C4 compounds (LPG). The line-up for LPG treating for hydrogen sulphide removal (and optionally carbon dioxide removal) is similar to the dry gas treatment, except for the presence of a liquid/liquid contactor instead of a gas absorber as the LPG fraction is liquid at the pressure used is the absorption process. Usually a packed or trayed contactor is used.

In some embodiments of the present invention, in the case of the formation of stable foams in the amine treating unit one or more defoaming agents may be added to the amine treater. Preferably the defoaming agent is chosen from silicone compounds, EO/PO based polyglycols and high boiling alcohols. Especially preferred are commercially available silicon based defoaming agents with the trade names NALCO EC9204, SAG 7133 and KS-604. Preferred polyglycol defoaming agents for use in aqueous systems are GE Betz's Maxamine 70B and Maxamine 82B and Nalco's EC 9079 A. The defoaming agent is suitably added to the recirculating amine stream, e.g. together with make-up amine solvent, or it is added directly to the amine solution or it may be sprayed onto the foam layer.

The chemical formulations of embodiments of this invention can be injected into the process streams under a wide range of temperature, pressure and phase conditions. These formulations can be adapted to various injection locations. The chemical formulations may be available in both aqueous and hydrocarbon phases. The chemical formulations are usually available in a wide range of concentrations.

Application of the process of the invention at least mitigates the formation of sour water emulsion and amine system emulsion and foams when cracking biofeed at the FCC. This will solve the waste water treatment plant operation problems (less emulsion to the waste water plant), enable the refinery to meet the quality specifications of the FCC products (better sulphur/CO2 removal), and reduce fresh amine replacement cost. The downstream FCC processes (product work-up) operate more stable and efficient than without the use of chemical additives according to the invention.

By breaking the emulsions and, if present, the foams in the oil/water separators, the sour water will not carry excess hydrocarbons to the downstream waste water treatment plant. Excessive hydrocarbon carry by the sour water can upset the waste water treatment plant and result in instable plant operation and higher sulfur oxide air emissions. Also the upset may result in increased chemical and biological oxygen demand (COD, BOD) which may threaten non-compliance of water discharge quality requirements.

By breaking the emulsions and foams in the amine treater, the amine wash and regenerator processes will continue to operate more efficiently so that product sulfur specification can be met and fuel gas can be processed without excessive sulfur oxide air emissions.

In a preferred embodiment, the one or more chemical additives selected from defoamers and demulsifiers for reducing or hindering the formation of foam in amine liquids are added to the streams entering the amine treater, to the amine solvent directly or to a make-up amine stream, or are sprayed onto the stable foam. The amount of chemical additive is suitably up till 1 vol % of the liquid product stream, preferably up till 0.1 vol %, more preferably up till 0.01 vol %, the minimum amount being at least 1 vppm, preferably 20 vppm.

The occurrence of foaming in an amine treater of an FCC unit is known. This problem occurs after prolonged operation of the amine unit. It is understood that this kind of foaming is due to contaminants derived from irreversible degradation of the base amine molecule itself. Further pollutants include solids/particulates, hydrocarbons and process chemicals. As indicated above the co-feed of biofuels resulted in the reversible formation of stable foams, which is clearly different than the above mentioned permanent foaming problems caused by prolonged operation. The use of the chemical additives in the amine treater according to the present invention prevents the formation of stable foam due to the co-processing of biofuel. In this respect it is observed that it is rather surprising that the (co)processing of biofeed results in the reversible formation of stable foams in the amine treaters, after all treating/separation steps in the product processing.

Some embodiments of the invention are further illustrated by the following non-limiting examples.

### EXAMPLES

### General

The amine used in the LPG (Liquefied Petroleum Gas) and dry gas washes was methyldiethanolamine (MDEA) in water, at concentrations of 25 wt % and 4.6 wt %, respectively. Accordingly, the nomenclature “LPG amine” and “dry gas amine” means in fact the MDEA solutions in water used to treat the LPG and dry gas, respectively. The “LPG amine” and dry gas amine” used in these foam and filtering tests were the same MDEA solutions as actually used in the amine treaters in the 3000 bbl/day fully integrated FCC unit.

The concentrations were determined by GC-MS and their water content by Karl Fischer analysis.

Foam Tests

The glassware was first cleaned with distilled water, rinsed with acetone and thoroughly dried before each experiment. 50 mL of amine sample was added to the glass gas washing cylinder. Nitrogen was bubbled at the given flow rates through the sample via a central glass tube fitted with frit reaching to the bottom of the glass cylinder. This created foaming of the amine. The foam level was allowed to stabilise at a certain height which was then read off for that nitrogen flow rate. The nitrogen flow was stopped, and the time measured for the foam to collapse so that there were no more bubbles in the amine solution. The time required is the “breaking down time” (Bt). These tests were done in duplicate for each amine sample. The average of the 2 measurements is reported.

Filtering Amine

The amine was filtered through a 0.45 μm Whatman FLHP filter to remove any suspended solids. The foam tests were then repeated as described above.

Anti-Foam Tests

A solution (2500 ppm in water) of fresh anti-foam agent Maxamine 70B from GE Betz was used. A working concentration of 5 ppm anti-foam agent in the amine was created by adding 100 μL of the above solution to 50 mL of the amine, which had been filtered through a 0.45 μm Whatman FLHP filter to remove any suspended solids.

The foam height and foam breakdown time were determined as described above.

The results for “LPG amine” are presented in Tables 1 and 2 and the results for “dry gas amine” in Table 3.

TABLE 1
“LPG amine” (Test run 6)
(Fh = Foam height; Bt = Breakdown time)

| | 5% fresh | | 25% fresh | | | | | | | | LPG Amine + | | |
|---|---|---|---|---|---|---|---|---|---|---|---|---|---|
| N₂ | MDEA | | MDEA | | | | | LPG Amine | | | 5 ppm anti | | |
| flow | in water | | in water | | LPG Amine | | | filtered | | | foam | | |
| rate | Fh | Bt | Fh | Bt | Fh | Fh | Bt | Fh | Fh | Bt | Fh | Fh | Bt |
| (L/h) | (mL) | (s) | (mL) | (s) | (mL) | (mm) | (s) | (mL) | (mm) | (s) | (mL) | (mm) | (s) |
| 0.0 | 54 | 0 | 52 | 0 | 56 | 50 | 0 | 51 | 45 | 0 | 52 | 46 | 0 |
| 22.8 | 66 | 9 | 58 | 2 | 66 | 63 | 3 | 60 | 85 | 2 | 58 | 86 | 1 |
| 37.5 | 126 | 23 | 118 | 40 | 86 | 76 | 100 | 96 | 95 | 71 | 70 | 97 | 38 |
| 68.3 | 230 | 25 | 146 | 40 | 88 | 73 | 95 | 94 | 80 | 64 | 78 | 87 | 42 |
| 91.0 | 255 | 29 | 158 | 41 | 86 | 68 | 80 | 92 | 70 | 60 | 84 | 82 | 45 |

The fresh 5 wt % MDEA in water has a higher foam height than 25 wt % solution.

It can be seen from the Table 1 above that the breakdown time of the LPG amine is progressively reduced in the order no treatment>filtered>+antifoaming agent.

TABLE 2
“LPG amine” (Test run 5)

| | | | LPG | | LPG Amine + 5 ppm | |
|---|---|---|---|---|---|---|
| N₂ | LPG Amine | | Amine filtered | | anti foam agent | |
| flow | Foam | Break- | Foam | | Foam | Breakdown |
| rate | height | down | height | Breakdown | height | time |
| (L/h) | (mL) | time (s) | (mL) | time (s) | (mL) | (s) |
| 0.0 | 52 | 0 | 52 | 0 | 52 | 0 |
| 22.8 | 60 | 2 | 70 | 18 | 74 | 3 |
| 37.5 | 190 | 60 | 160 | 41 | 118 | 28 |
| 68.3 | 190 | 55 | 132 | 50 | 140 | 30 |
| 91.0 | 200 | 70 | 120 | 52 | 138 | 40 |

It can be seen from the Table 2 above that the breakdown time of the LPG amine is progressively reduced in the order no treatment>filtered>+antifoaming agent.

The foam height is also significantly reduced.

TABLE 3
“Dry gas amine” (Test Run 6)

| | | | | | Dry Gas Amine + 5 | |
|---|---|---|---|---|---|---|
| | Dry gas | | Dry Gas Amine | | ppm anti-foaming | |
| N2 | Amine | | filtered | | agent | |
| flow | Foam | Break- | Foam | | Foam | |
| rate | height | down | height | Breakdown | height | Breakdown |
| (L/h) | (mL) | time (s) | (mL) | time (s) | (mL) | time (s) |
| 0.0 | 52 | 0 | 53 | 0 | 53 | 0 |
| 22.8 | 90 | 34 | 110 | 50 | 56 | 4 |
| 37.5 | 170 | 55 | 260 | 60 | 150 | 26 |
| 68.3 | 190 | 65 | 300 | 60 | 160 | 28 |
| 91.0 | 200 | 75 | 320 | 70 | 170 | 29 |

Although filtering has little positive effect, the use of 5 ppm of the antifoaming agent has a significant effect on reducing the breakdown time. The use of the antifoaming agent also significantly reduces the foam height.

Therefore, embodiments of the present invention are well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, substituted, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount whether accompanied by the term “about” or not. In particular, the phrase “from about a to about b” is equivalent to the phrase “from approximately a to b,” or a similar form thereof. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

1. A process for the fluid catalytic cracking of oxygenated hydrocarbon compounds from biological origin, the process comprising
a) contacting a feed comprising the oxygenated hydrocarbon compounds with a fluid catalytic cracking catalyst at elevated temperature to produce a cracked products stream, the feed comprising an amount of sulphur;
b) separating catalyst from the cracked products stream;
c) separating a light fraction from the cracked products stream; and
d) removing hydrogen sulphide from the light fraction by means of an amine treating process;
wherein the fluid catalytic cracking process involves the presence or use of water and/or steam, and
wherein the fluid catalytic cracking process furthermore comprises a working-up process of the cracked products stream, in which working-up process one or more chemical additives for reducing or hindering the formation of foam in amine liquids selected from defoamers and demulsifiers are added to the amine solvent in one or more amine treaters.

2. The process of claim 1, wherein the oxygenated hydrocarbon compounds are derived from oil and fats from plant sources, animal sources or microbial sources, preferably tri-glycerides and/or free fatty acids.

3. The process of claim 1, wherein the amount of oxygenated hydrocarbon compounds is up to 65 vol % of the total feed.

4. The process of claim 1, wherein the amount of oxygenated hydrocarbon compounds is between 1 and 45 vol %.

5. The process of claim 1, wherein the amount of oxygenated hydrocarbon compounds is between 2 and 35 vol %.

6. The process of claim 1, wherein the amount of oxygenated hydrocarbon compounds is between 3 and 25 vol %.

7. The process of claim 1, wherein the amount of sulphur in the feed is up to 4 wt % based on total feed.

8. The process of claim 1, wherein the amount of sulphur in the feed is up to 3 wt %.

9. The process of claim 1, wherein the amount of sulphur in the feed is between 0.1 and 2.5 wt %.

10. The process of claim 1, wherein in step a) the elevated temperature is in the range of 300 to 750° C. and/or the contact time between the feed and the fluid catalytic catalyst is less than 10 seconds.

11. The process of claim 1, wherein the light fraction from the cracked products stream is a C1-C2 fraction or a C3-C4 fraction.

12. The process of claim 1, wherein the light fraction from the cracked products stream is obtained by feeding separated cracked products stream to a distillation column and fractionating the cracked products stream into an offgas fraction comprising C1-C4 compounds and at least one further fraction.

13. The process of claim 12 followed by separating the fraction comprising the C1-C4 fraction into a fraction comprising C1-C2 compounds and a fraction comprising C3-C4 compounds.

14. The process of claim 11 wherein hydrogen sulphide is removed from a fraction comprising C1-C2 compounds and/or from a fraction comprising C3-C4 compounds.

15. The process of claim 1, wherein steam is added to the feed/fluid cracking catalyst and/or steam is used to improve the separation of the catalyst from the cracked products stream.

16. The process of claim 1, wherein the light fraction is cooled down to obtain a cooled down gas stream and a liquid oil/water condensate, followed by separation of the oil and the water fraction in an oil/water separator.

17. The process of claim 1, wherein the cooled down gas stream, before the further separation, is compressed to a pressure between 0.5 and 5 MegaPascal, whereafter the compressed gas stream is cooled down to obtain a cooled down gas stream and a liquid oil/water condensate, followed by separation of the oil and the water fraction in an oil/water separator.

18. The process of claim 11, wherein an obtained fraction comprising C3-C4 compounds is cooled down to obtain a cooled down gas stream and a liquid oil/water condensate, followed by separation of the oil and the water fraction in an oil/water separator.

19. The process of claim 16, wherein one or more chemical additives for separating oil/water emulsions into oil and water selected from demulsifiers and defoamers are added to the streams entering the oil/water separator or to the emulsions in the oil/water separator.

20. The process of claim 19, wherein the chemical additive for separating oil/water emulsions into oil and water is chosen from (alkyl)phenol-formaldehyde resins, epoxy resins, amines, polyamines, amides, di-epoxides, alcohols, polyols, polyol block copolymers, and the alkoxylated, especially ethoxylated or propoxylated, derivatives there from.

21. The process of claim 1, wherein the one or more chemical additives for reducing or hindering the formation of foam in amine liquids selected from defoamers and demulsifiers are chosen from silicone compounds, EO/PO based polyglycols and high boiling alcohols.

22. The process of claim 1, wherein the one or more chemical additives selected from defoamers and demulsifiers for reducing or hindering the formation of foam in amine liquids are added to the streams entering the amine treater, to the amine solvent directly or to a make-up amine stream, or are sprayed onto the stable foam.
